(19) United States
(12) Patent Application Publication
(10) Pub. No.: US 2022/0265827 A1
(43) Pub. Date: Aug. 25, 2022

(54) FUNCTIONAL MICROSCAFFOLD THAT CAN BE MAGNETICALLY ACTUATED AND MANUFACTURING METHOD THEREFOR

(71) Applicant: BIOT KOREA INC., Gwangju (KR)

(72) Inventors: Yeong Jun CHANG, Seoul (KR); Bo Bae CHO, Seoul (KR); Seul Ki LEE, Seoul (KR)

(21) Appl. No.: 17/662,923

(22) Filed: May 11, 2022

Related U.S. Application Data
(63) Continuation of application No. PCT/KR2020/016394, filed on Nov. 19, 2020.

(30) Foreign Application Priority Data
Nov. 21, 2019 (KR) 10-2019-0150529

(51) Int. Cl. A61K 41/00; A61K 47/02

(52) U.S. Cl. CPC A61K 41/0052; A61K 47/02

(57) ABSTRACT

This application relates to a magnetically actuatable functional microscaffold for efficient targeted therapy of intractable diseases and a preparation method thereof. The magnetically actuatable functional microscaffold and the preparation method can enable the application of magnetic nanoparticles according to the status of the scaffold and enable various types of targeted therapy in the field of targeted therapy of intractable diseases.

### CROSS-REFERENCE TO RELATED APPLICATIONS

This application is a continuation application, and claims the benefit under 35 U.S.C. § 120 and § 365 of PCT Application No. PCT/KR2020/016394 filed on Nov. 19, 2020, which claims priority to Korean Patent Application No. 10-2019-0150529 filed on Nov. 21, 2019, the contents of each of which are hereby incorporated by reference in their entirety.

### BACKGROUND

### Technical Field

The present disclosure relates to a magnetically actuatable functional microscaffold and, more particularly, to a customized method of bonding magnetic nanoparticles having a positively or negatively charged surface to biodegradable microscaffolds, for efficient targeted therapy of intractable diseases.

### Description of Related Technology

A scaffold is an artificially created extracellular matrix (ECM) for tissue construction and control of cell functions, and is a structural tissue support acting as a cell adhesion inducer. These scaffolds must be designed in consideration of properties thereof such as biocompatibility, degradability, porosity, water content, transport, and cell adhesion. Recently, magnetic properties of the scaffolds have been paid attention.

A scaffold containing magnetic particles attracts ex vivo growth factors and biomaterials in vivo, and increases the rates of bone cell growth and differentiation. Thus, a method for manufacturing a magnetic polymer scaffold for bone regeneration has been reported (refer to Korea Patent Application Publication Nos. 10-2016-0047819, 10-2016-0031682, and 10-2016-0031683). present disclosure

### SUMMARY

The inventors of the present disclosure have attempted to manufacture a functional microscaffold capable of supporting therapeutic substances such as cell therapy agents and drugs and of being magnetically actuated by an external driving device in order to enable more efficient targeted therapy while having the advantageous characteristics of existing studies. In addition, the inventors have attempted to provide a suitable bonding method specific to the surface property of magnetic nanoparticles.

While conducting research on microscaffolds that can be magnetically actuated by an external driving device for efficient targeted therapy of intractable diseases, the inventors of the present disclosure have found a customized method of bonding positively or negatively charged magnetic nanoparticles with biodegradable microscaffolds and have confirmed that the prepared microscaffolds were actuated by an external driving device, indicating that non-invasive and effective targeted therapy is possible.

Therefore, the objective of the present disclosure is to provide a functional microscaffold that enables efficient targeted therapy of intractable diseases by responding to an external driving device and to provide a customized scaffold bonding method according to the surface potential of magnetic nanoparticles (MNP).

In one aspect of the present disclosure, there is provided a functional microscaffold for targeted therapy, the microscaffold being composed of a biodegradable polymer and being loaded with magnetic particles in a three-dimensional porous microstructure thereof or on the surface of the three-dimensional porous microstructure thereof.

In one embodiment, the magnetic particles may be nanoparticles that are positively or negatively charged. The positively charged magnetic nanoparticles may include a cationic polymer compound, and the negatively charged magnetic nanoparticles may be prepared through reaction with an acidic material.

In another aspect of the present disclosure, there is provided a method of preparing the functional microscaffolds for targeted therapy, the method including: (a) reacting iron chloride with a cationic high molecular compound or an acidic material to obtain positively charged magnetic nanoparticles or negatively charged magnetic nanoparticles; and (b) loading the positively charged magnetic nanoparticles or negatively charged magnetic nanoparticles obtained in step (a) onto porous microstructures made of a biodegradable polymer.

According to the present disclosure, it was discovered that the present disclosure can be applied to a wide area of diseases by bonding positively or negatively charged magnetic nanoparticles with microscaffolds through a customized bonding method and by confirming that the manufactured functional scaffolds are normally actuated by an external driving device.

Accordingly, the magnetically actuatable functional microscaffold of the present disclosure and the preparation method thereof are technologies enabling the application of magnetic nanoparticles according to the status of the scaffold and enabling targeted therapy of intractable diseases in the field of targeted therapy of intractable diseases.

### BRIEF DESCRIPTION OF DRAWINGS

FIGS. 1A to 1C are of manufactured scaffolds.

FIG. 2 schematically illustrate a bonding reaction between PEI-MNP and scaffolds.

FIG. 3 shows photographs of PEI-MNP scaffolds obtained by bonding various concentrations of PEI-MNPs and scaffolds, with or without a coupling agent.

FIG. 4 is an SEM image illustrating proximity surface conditions of PEI-MNP-coated scaffolds having various sizes (200 to 400 μm, and 400 μm or more).

FIGS. 5A to 5D are SEM-EDS results confirming the presence of Fe on the surface of the PEI-MNP-coated scaffolds.

FIG. 6 schematically illustrates a bonding reaction between CA-MNP and scaffolds.

FIG. 7 is a result of binding CA-MNP and scaffold using a water-dispersed scaffold.

FIG. 8 is a result of binding CA-MNP and scaffold using a dried scaffold.

FIG. 9 is a SEM photograph showing the proximity surface condition of a CA-MNP-coated scaffold.

FIGS. 10A to 10C are SEM-EDS results confirming the presence of Fe on the surface of the CA-MNP-coated scaffolds.

FIG. 11 schematically illustrates a bonding reaction between Feraheme and a scaffold.

FIG. 12 is a comparison result of the magnetic strength of the scaffold combined with each nanoparticle (PEI-MNP, CA-MNP and Feraheme).

FIG. 13 is a photograph of a state in which a PEI-MNP-coupled microscaffold and a CA-MNP-coupled microscaffold are driven by an external driving device (EMA).

### DETAILED DESCRIPTION

The present disclosure provides a functional microscaffold for targeted therapy, the microscaffold being composed of a biodegradable polymer and being loaded with magnetic particles in a three-dimensional porous microstructure or on the surface of the three-dimensional porous microstructure.

In one embodiment, the magnetic particles may be positively charged magnetic nanoparticles. Preferably, the magnetic particle may be an iron compound having a positive charge (for example, iron chloride), but may not be limited thereto. In addition, to obtain the positively charged magnetic nanoparticles, a cationic high molecular compound such as polyethyleneimine may be used to impart a positive charge to the nanoparticles, but the preparation method may not be limited thereto.

In one embodiment, the magnetic particles may be negatively charged magnetic nanoparticles. Preferably, the magnetic particle may be an iron compound having a negative charge (for example, iron chloride or commercially available iron compounds), but may not be limited thereto. In addition, the negatively charged magnetic nanoparticles may be prepared by reacting an iron compound with an acidic substance such as citric acid to impart a negative charge to the nanoparticles, but the preparation method of the negatively charged magnetic nanoparticles may not be limited thereto.

The functional microscaffold for targeted therapy may be manufactured by a method including: (a) reacting iron chloride with a cationic high molecular compound or an acidic material to obtain positively charged magnetic nanoparticles or negatively charged magnetic nanoparticles; and (b) loading the positively charged magnetic nanoparticles or negatively charged magnetic nanoparticles obtained in step (a) onto a porous microstructure composed of a biodegradable polymer.

The preparation method of the present disclosure includes the step of obtaining positively charged magnetic nanoparticles or negatively charged magnetic nanoparticles by reacting iron chloride with a cationic high molecular compound or an acidic substance [i.e., step (a)]. In step (a), the cationic high molecular compound or acidic material is the same as described above. In the case of reacting the iron chloride with the acidic material in step (a), nanoparticle nuclei are formed by stirring an aqueous solution containing water and the iron chloride, a basic material (for example, aqueous ammonia, etc.) is added dropwise to the aqueous solution to grow the nanoparticle nuclei, and an acidic material such as citric acid is added thereto to form negatively charged magnetic nanoparticles.

The manufacturing method of the present disclosure includes the step of loading the positively charged magnetic nanoparticles or negatively charged magnetic nanoparticles obtained in step (a) onto a porous microstructure composed of a biodegradable polymer (for example, step (b)). In step (b), the porous microstructure composed of a biodegradable polymer can be prepared by modifying the method described in Korean Patent Application No. 10-2018-0044095.

In the case of loading positively charged magnetic nanoparticles on porous microstructures, an EDC/NHS coupling reaction may be used. Alternatively, the loading may be performed by electrostatic bonding between the positively charged surface of positively charged magnetic nanoparticles and the negatively charged surface of microstructures (i.e., microscaffolds).

When loading negatively charged magnetic nanoparticles on porous microstructures, the surface of the negatively charged microstructures (microscaffolds) is modified with a cationic polymer so that the microstructures have a positively charged surface, and the negative charges (for example, a carboxyl group, etc.) on the negatively charged magnetic nanoparticles are activated with a coupling agent or the like, and then a coupling reaction between the microstructures having the positively charged surface obtained through surface modification and the nanoparticles having the activated negatively charged surface is carried out.

Alternatively, the negatively charged magnetic nanoparticles are first reacted with a cationic polysaccharide such as chitosan and then the nanoparticles are attached to the negatively charged surface of the microstructures (microscaffolds). In this manner, the negatively charged nanoparticles can be loaded on the porous microstructures.

Hereinbelow, the present disclosure will be described in more detail with reference to examples and experimental examples. However, the examples and experimental examples are presented only for illustrative purposes and thus should not be interpreted to limit the scope of the present disclosure.

### Example

### 1. Preparation of Magnetic Nanoparticles

(1) Preparation of Magnetic Nanoparticles (PEI-MNP) with Positively Charged Surface

48 ml of a 1 M hydrochloric acid solution in which 2.536 g (0.02 mol) of FeCl₂ and 6.488 g (0.04 mol) of FeCl₃ were dissolved was slowly added to 200 ml of a 1 M sodium hydroxide solution and stirred at 1,500 rpm in a nitrogen atmosphere. A reaction was carried at 80° C. for 2 hours, and then 20 ml of distilled water in which 12 g of polyethyleneimine (branched, PEI, [MW 10,000]) was dissolved was added and stirred at 95° C. for 30 minutes. After completion of the reaction, the precipitate was washed three times with distilled water at about 40° C. and dispersed in 100 ml of distilled water.

(2) Preparation of Magnetic Nanoparticles (CA-MNP) with Negatively Charged Surface

In a nitrogen atmosphere, 3.464 g (0.016 mol) of FeCl₂.4H₂O and 8.88 g (0.0032 mol) of FeCl₂.6H₂O were dissolved in 200 ml of distilled water and stirred at 1,000 rpm for 20 minutes to generate nanoparticle nuclei. 50 ml of aqueous ammonia was slowly dropped, and the temperature was slowly raised so that the reaction was performed at 80° C. for 30 minutes to grow the nuclei. Then, a solution obtained by dissolving 4 g of citric acid in 8 ml of distilled water was added, the temperature was raised to 90° C., and the reaction was performed for 1 hour, followed by cooling to room temperature. The supernatant was removed using magnetic properties, and about 200 ml of distilled water was added to the nanoparticle solution and centrifuged at 10,000 rpm for 10 minutes to remove the supernatant. About 200 ml of distilled water was added to the separated precipitate again, and the precipitate was dispersed with an ultrasonicator and centrifuged at 10,000 rpm for 10 minutes to recover the supernatant.

### 2. Preparation of Scaffold

poly(lactic-co-glycolic acid (PLGA) microscaffolds were prepared by modifying the method described in Korean Patent Application No. 10-2018-0044095. The summary is given below. Poly(lactic-co-glycolic acid) (PGLA) scaffolds were prepared through a double emulsion method using a microfluidic device composed of a Tygon tube ( 1/32, 1/16, 3/32 in d.d), a 21G needle, and a tubing adapter. First, PLGA and a gelatin solutions were prepared for water-in-oil (W-O) emulsification. Specifically, 210 mg of PLGA was dissolved in a solution containing 2.8 ml of dichloromethane (DCM) and 45 μl of Span80 under the condition of 3000 rpm for 7 minutes. Next, 1750 μl of a gelatin solution (0.3 g, 4.7 ml polyvinyl alcohol (PAV) 1% aqueous solution) was added to the PLGA solution to make a W-O emulsion, and the mixture was stirred at 3000 rpm for 4 minutes and 30 seconds.

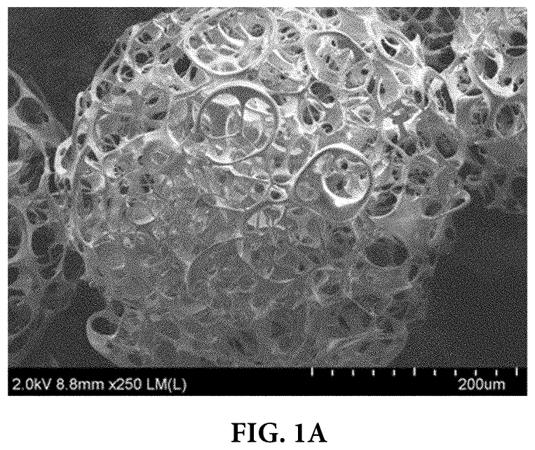

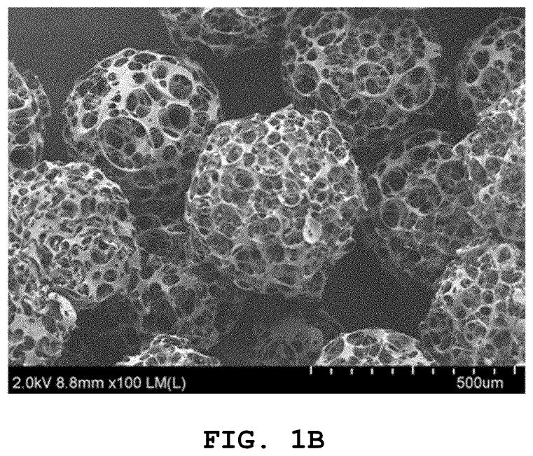

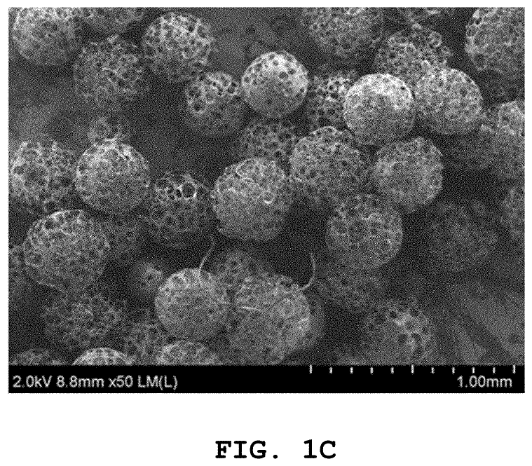

The emulsified W-O solution was transferred to a 20 ml vial, a tubing adapter of a 26G tube and a Tygon tube was connected to the vial, and a 21G needle was inserted into a portion through which the final scaffold was discharged, the microfluidic device was operated at a pressure of 30 to 24 kpa in a nitrogen gas state. The W-O emulsion and 1% PVA aqueous solution, were continuously injected so that the microscaffolds were continuously discharged through the Tygon tube of the microfluidic device. Next, the scaffolds flowed along the 21G needle connected to the tubing adapter were collected in deionized water contained in a 2 L double beaker maintained at constant temperature. Then, in order to remove the dichloromethane (DCM) contained in the collected scaffolds, the scaffold solution was gently stirred over 4 hours to allow the evaporation of the DCM. Thus, PLGA microparticles containing gelatin were obtained. Thereafter, in order to remove the gelatin contained in the scaffolds, the gelatin microparticles were immersed in deionized water contained in a 1000 ml beaker (38° C.) and stirred for 5 minutes. Finally, the resulting product was washed 5 times with deionized water, resulting in gelatin-leached PLGA microscaffolds contained in in the 20 ml vial containing deionized water (see FIGS. 1A-1C)

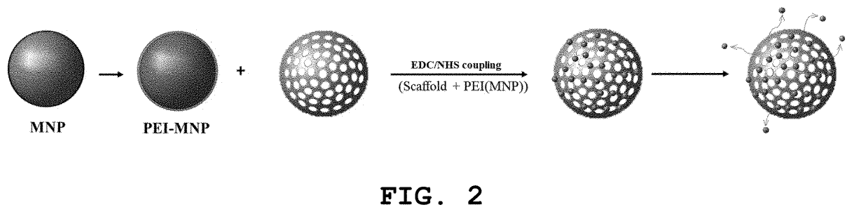

3. Binding Reaction between Magnetic Nanoparticles and Scaffolds
(1) Bonding Reaction between Magnetic Nanoparticles and Scaffolds on Positively Charged Surface (See FIG. 2)

The prepared PEI-MNP solution was diluted to concentrations of 60 mg/ml, 30 mg/ml, 15 mg/ml, 7.5 mg/ml, 3.75 mg/ml, 1.875 mg/ml, 0.9375 mg/ml, 0.46875 mg/ml. The prepared PLGA scaffold (water dispersion state: addition of 0.5 ml and removal of distilled water (DW), dry state: 0.004) was added to 2.5 ml of a coupling solution obtained by dissolving 0.2875 g of 1-ethyl-3-(3-dimethylaminopropyl) carbodiimide hydrochloride (EDC) and 0.1725 g of N-hydroxysuccinimide (NHS) in 5 ml of 0.1 M 4-morpholinoethanesulfonic acid (MES), followed by vortexing for 6 hours to activate the surface of the scaffold. 2.5 ml of the PEI-MNP solution having each concentration prepared above was added to the solution in which the scaffold was dispersed, and the mixture was stirred for about 16 hours. In addition, in order to check the electrostatic bonding between the positively charged surface of the PEI-MNP and the negatively charged surface of the scaffold without a coupling agent, 2.5 ml of the MNP solution diluted to each of the described concentrations was added to 2.5 ml of the distilled water (DW) in which the scaffolds were dispersed for about 16 hours, followed by stirring.

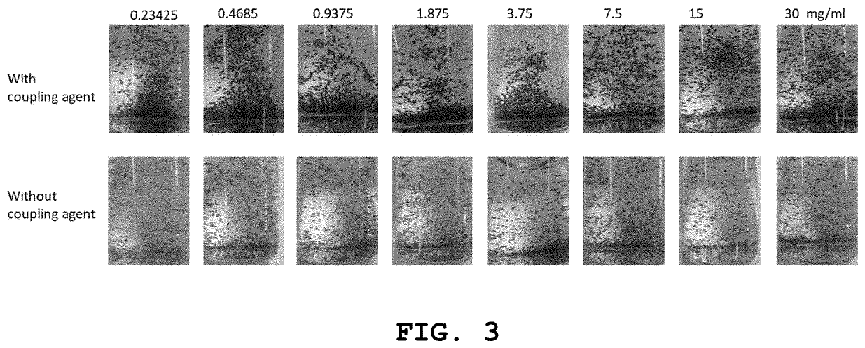

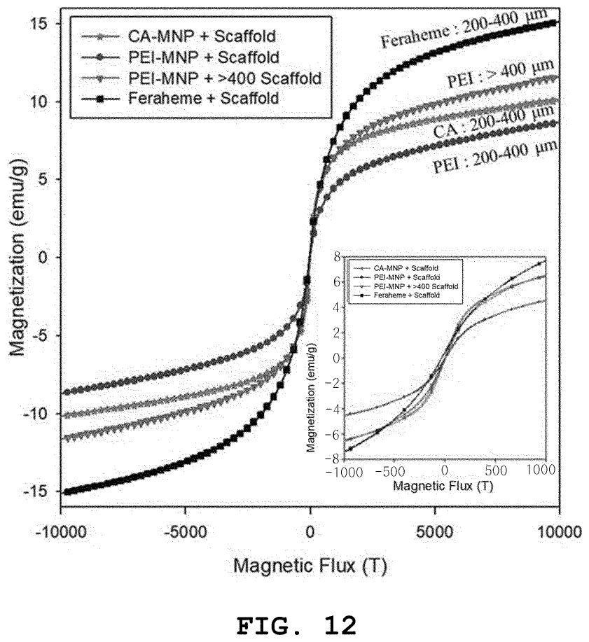

In this case, the final concentrations of the MNP solution were 30 mg/ml, 15 mg/ml, 7.5 mg/ml, 3.75 mg/ml, 1.875 mg/ml, 0.9375 mg/ml, 0.46875 mg/ml, and 0.23425 mg/ml (see FIG. 3). After the reaction, the PEI-MNP scaffolds were washed with deionized water through a cell strainer (100 mesh), and the washed scaffolds were collected. For the scaffolds with sizes of 200 to 400 and the scaffolds with sizes of larger than 400 μm, the same process as described above was performed, and then the magnetic strength of the scaffolds with sizes of 200 to 400 and the magnetic strength of the scaffolds with sizes of larger than 400 μm were compared (see FIG. 12).

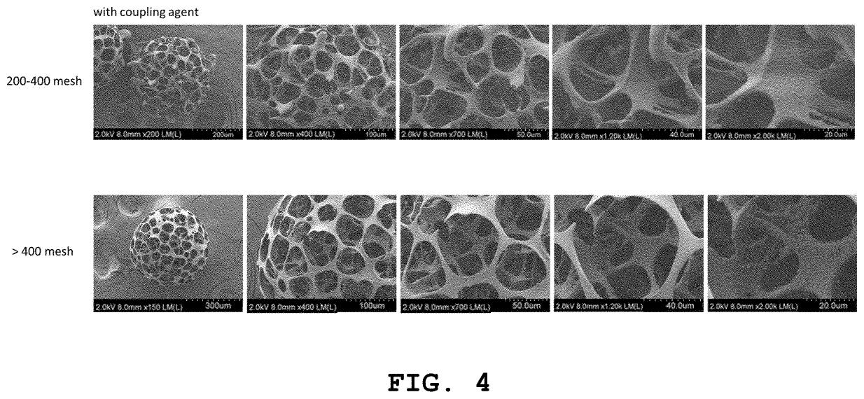

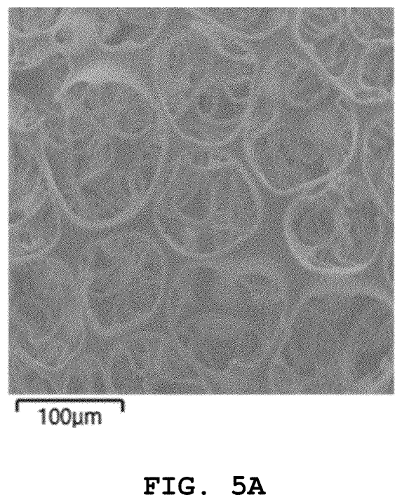

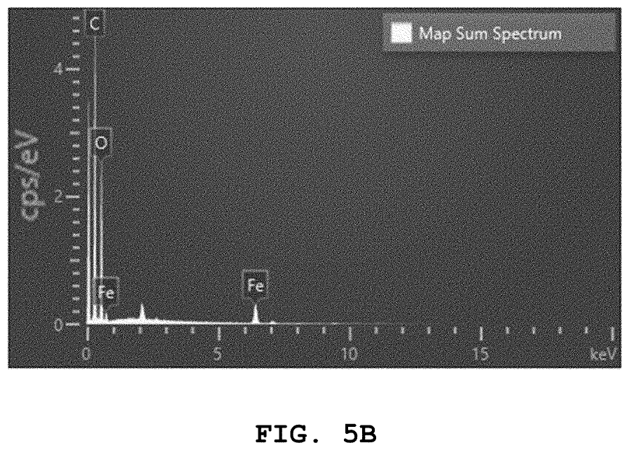

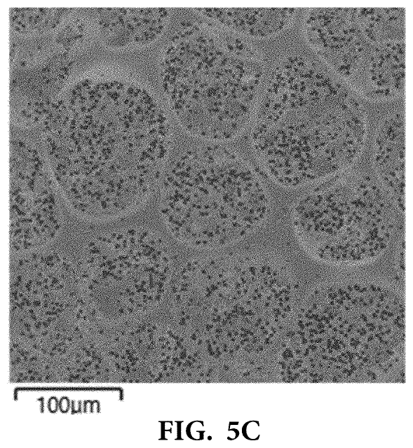

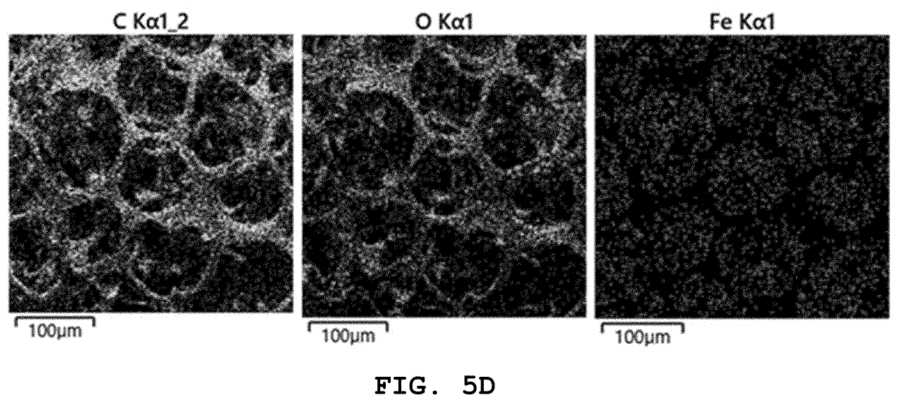

The surface of the scaffolds bound to the PEI-MNP was observed through SEM measurement, and the presence of Fe, which is a component of the PEI-MNP applied to the scaffold, was confirmed through surface mapping of SEM-EDS (FIGS. 4 and 5A to 5D).

(2) Bonding Reaction between Magnetic Nanoparticles and Scaffolds on Negatively Charged Surface
(2-1) Bonding between CA-MNP and Scaffold

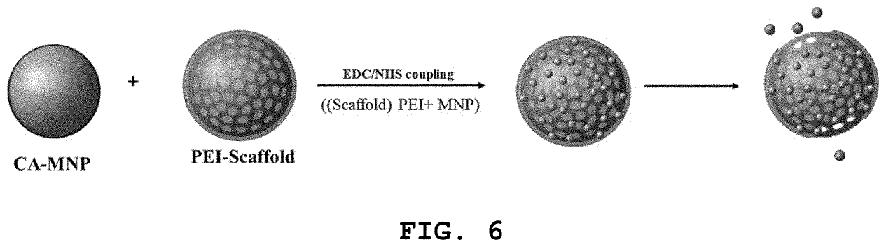

FIG. 6 schematically illustrates a bonding reaction between CA-MNP and scaffolds.

### (i) Preparation of PEI-Coated Scaffold

0.03 g of the lyophilized scaffold was added to 10 ml of PEI solutions diluted to concentrations of 10%, 20%, 30%, 40%, and 50%, respectively, and stirred (vortexed) for 18 hours. 50 ml of the aqueous scaffold dispersion was added to 500 ml of each of 1%, 5%, and 10% diluted PEI solutions slowly, and stirred for 18 hours to coat the surface of the negatively charged scaffold with the PEI, which is a cationic polymer, so that the surface of the scaffold was modified to be positively charged. After completion of the reaction, the scaffolds were sufficiently washed with distilled water (DW) and lyophilized to produce PEI-coated scaffolds.

### (ii) CA-MNP Activation

3 ml (6 mg/ml, 3 ml→18 mg) of the CA-MNP prepared in the process described above was added to 5 ml of a coupling solution obtained by dissolving 0.2875 g of EDC and 0.1725 g of NHS in 0.1 M MES buffer and stirred (vortexed) to activate the carboxyl groups (—COOH) on the surface of the CA-MNP.

(iii) Bonding of Activated CA-MNP and PEI Scaffold

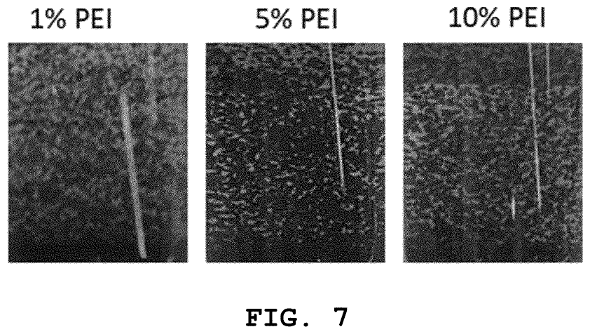

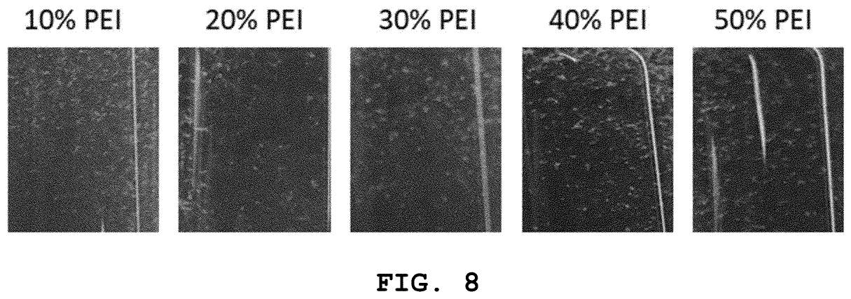

0.02 g of the prepared PEI-coated scaffold was added to the activated CA-MNP solution and stirred for 15 hours to induce a coupling reaction. After completion of the reaction, the reaction product was sufficiently washed with distilled water (DW) and lyophilized, and the CA-MNP scaffolds were collected (see FIGS. 7 and 8).

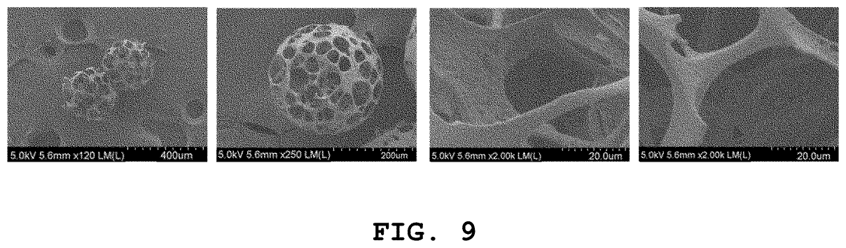

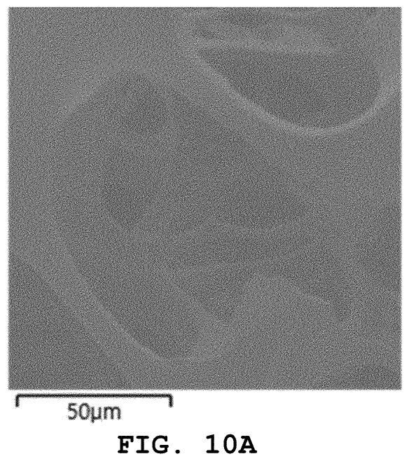

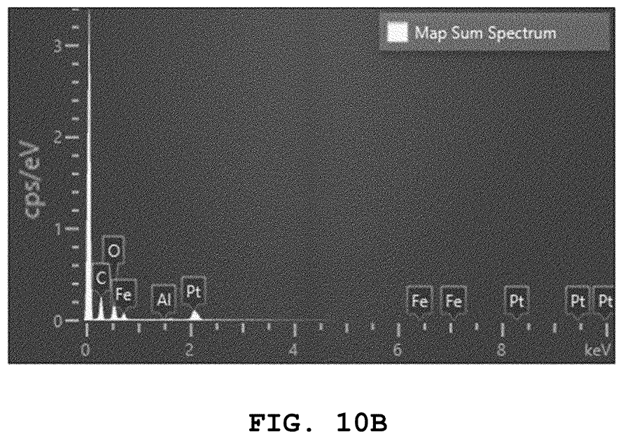

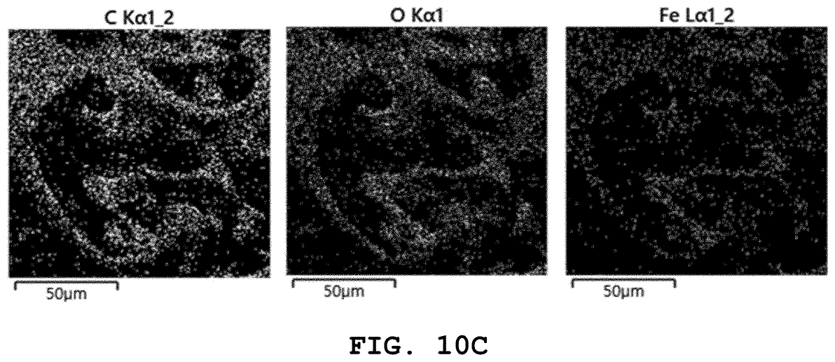

The surface of the CA-MNP scaffolds prepared through the reaction was observed through SEM measurement, and the presence of Fe, which is a component of the CA-MNP applied to the scaffolds, was confirmed through surface mapping of SEM-EDS (see FIG. 9 and FIGS. 10A to 10C).

### (2-2) Bonding of Feraheme and Scaffold

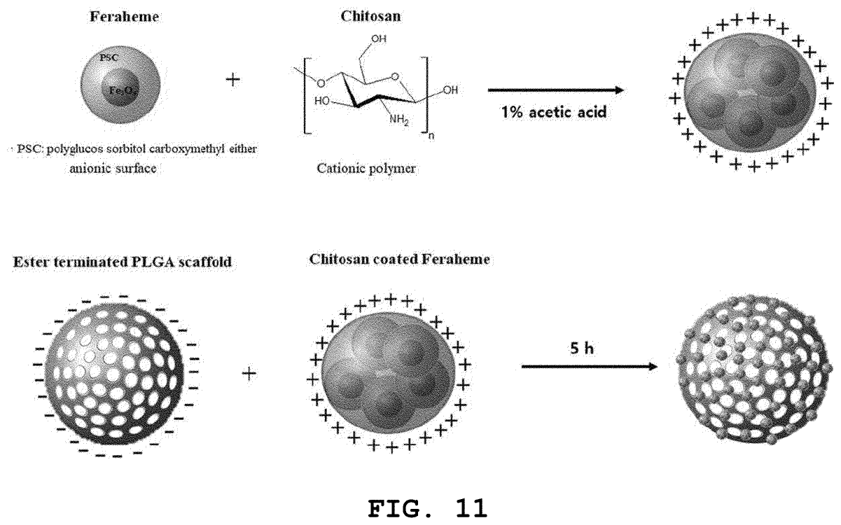

Feraheme (approved by FDA and manufactured by Amag pharmaceuticals Inc.) is an iron nanoparticle having a negatively charged surface. The Feraheme was reacted with chitosan, which is a cationic polysaccharide, so that the adsorption power thereof to the scaffold having a negatively charged surface was enhanced (see FIG. 11).

### (i) Bonding of Feraheme and Chitosan

For a bonding reaction with the scaffold, Feraheme, which is a commercially available anemia treatment drug, was used. A chitosan solution was prepared by dissolving 40 mg of chitosan in 60 ml of a 1% acetic acid solution. 3 ml of Feraheme (30 mg/ml) was added to the chitosan solution, stirred (vortexed) for 3 hours, washed thoroughly, and centrifugated to remove residual acetic acid. After redispersing by adding 3 ml of distilled water (DW), the concentration of Fe was measured through ICP analysis to obtain a Feraheme chitosan solution having a concentration of 20 mg/ml.

### (ii) Bonding of Chitosan-Treated Feraheme and Scaffolds

The prepared Feraheme chitosan solution (3.75 mg Fe) was added to an e-tube containing the scaffold (water dispersion state: obtained by filling the tube with 0.5 ml of the scaffold and removing distilled water, dry state: 0.01 g), and fixed for 5 or more hours to induce a surface adsorption reaction. The adsorption power of the surface Feraheme chitosan was enhanced through lyophilization, unreacted Feraheme chitosan particles were removed with a particle separator, and MNP scaffolds with sizes in the range of 300 to 400 μm were collected.

### Experimental Example

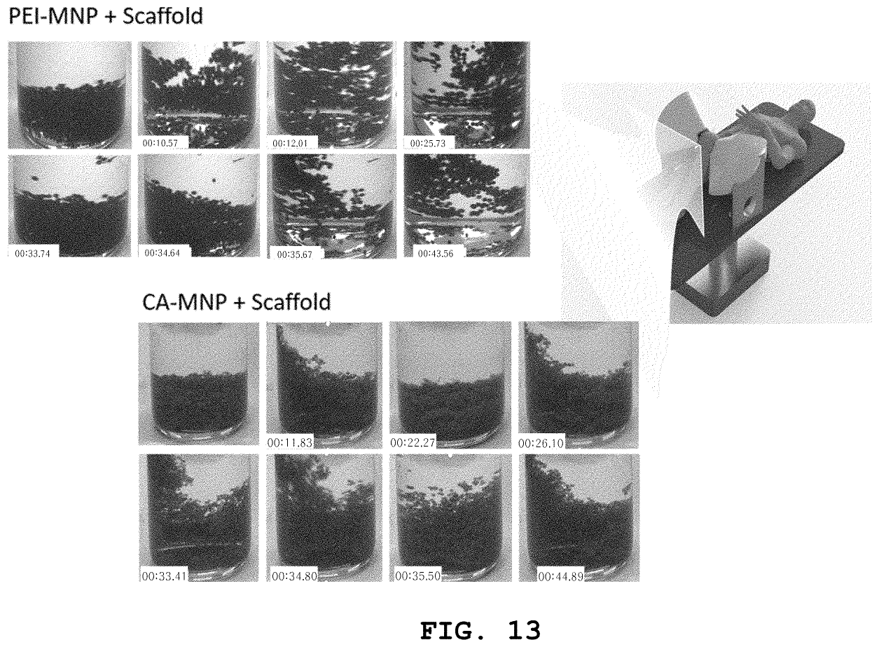

The PEI-MNP-coupled microscaffold and the CA-MNP-coupled microscaffold were actuated with an electro magnetic actuator (EMA), which is an external driving device, to check the possibility of targeted therapy (see FIG. 13).

According to the present disclosure, it is turned out that the present disclosure can be applied to a wide area of diseases by bonding positively or negatively charged magnetic nanoparticles with microscaffolds through a customized bonding method and by confirming that the manufactured functional scaffolds are normally actuated by an external driving device. Accordingly, the magnetically actuatable functional microscaffold of the present disclosure and the preparation method thereof are technologies enabling the application of magnetic nanoparticles according to the status of the scaffold and enabling various targeted therapies, thereby being usefully applied in the related industry.

What is claimed is:

1. A functional microscaffold for targeted therapy, the microscaffold comprising a biodegradable polymer and having a three-dimensional porous structure loaded with magnetic particles inside thereof or on a surface thereof.

2. The functional microscaffold of claim 1, wherein the magnetic particles are positively charged magnetic nanoparticles or negatively charged magnetic nanoparticles.

3. The functional microscaffold of claim 2, wherein the positively charged magnetic nanoparticles comprise cationic high molecular compounds.

4. The functional microscaffold of claim 2, wherein the negatively charged magnetic nanoparticles are configured to be prepared through reaction with an acidic material.

5. A method of preparing the functional microscaffold for targeted therapy of claim 1, the method comprising: reacting iron chloride with a cationic high molecular compound or an acidic material to obtain positively charged magnetic nanoparticles or negatively charged magnetic nanoparticles; and loading the obtained positively charged magnetic nanoparticles or negatively charged magnetic nanoparticles onto porous microstructures comprising a biodegradable polymer.
